(19) United States
(12) Patent Application Publication
(10) Pub. No.: US 2004/0256172 A1
(43) Pub. Date: Dec. 23, 2004

(54) Shape-retaining stethoscope tubing

(76) Inventor: Dean William Darling, Sauk City, WI (US)

Correspondence Address: Robert S. Schiller, 520 27th Street, Sacramento, CA, 95816, US

(21) Appl. No.: 10/601,506

(22) Filed: Jun. 23, 2003

(51) Int. Cl. A61B 7/02

(52) U.S. Cl. 181/131

(57) ABSTRACT

A stethoscope tubing with a means for retaining a desired shape, said means being a wire or plurality of wires integrated into flexible acoustic stethoscope tubing. Also, for retrofit of existing stethoscopes, a sleeve with a means for retaining a desired shape is used.

### BACKGROUND—FIELD OF INVENTION

This invention relates to stethoscopes, specifically to an improvement in the flexible acoustic tubing between the sound gathering chestpiece and the binaural eartubes.

### BACKGROUND—DESCRIPTION OF PRIOR ART

Stethoscopes are essential diagnostic instruments used in daily medical practice. Most have two earpieces and rigid binaural eartubes connected at a Y-shaped juncture, with one or two lengths of flexible acoustic tubing leading to a sound gathering chestpiece. In a medical situation, typically the user of the stethoscope holds the chestpiece in his or her hand and places it against the bare skin of the person being examined. While this works well in a clinic or hospital environment, in emergency on-site locations this method is not always preferable or possible due to the desire to keep the victim's clothing in place. For example, in an automobile accident in cold weather complications such as hypothermia or shock may arise from removing clothing from a victim trapped in wreckage. In addition, further damage may occur to the victim by forcibly removing clothing for stethoscope access.

U.S. Pat. No. 2,807,328 to Gould (1954) addresses this shortcoming by means of a rigid tube to allow the user to pass the chestpiece of the stethoscope beneath the clothing of the person being examined; however, this design, by its rigidity, does not allow for flexible placement of the chestpiece, nor does it lend itself easily to retrofit of existing stethoscopes. U.S. Pat. No. 5,952,618 to Deslauriers (1999) provides stethoscopic tubing with an improved acoustic conduit and a spring for self-straightening, non-kinking operation; however, this tubing does not retain its shape when bent. While these and other prior inventions may have merit, they do not address the problem mentioned above, namely, allowing medical specialists the ability to quickly, flexibly and accurately position a stethoscope chestpiece under clothing in emergency situations.

### OBJECTS AND ADVANTAGES

Accordingly, several objects and advantages of the present invention are:

(a) to provide a stethoscope tubing which can be bent to conform to a desired shape and which will retain its shape for placement beneath the clothing of the patient without interfering with the stethoscope's operation;

(b) to provide a shape-retaining stethoscope tubing which can be manufactured into new stethoscopes or which can be retrofit onto existing stethoscopes of both the single and double tube type;

(c) to provide a shape-retaining stethoscope tubing which can allow the stethoscope to be stored about the neck of a medical professional without falling or swinging.

Further objects and advantages of my invention will become apparent from a consideration of the drawings and ensuing description.

### SUMMARY

In accordance with the present invention a stethoscope tubing comprises a wire or wires integrated into the flexible acoustic tubing of an existing stethoscope or into a separate flexible sleeve for retrofit of an existing stethoscope.

### DRAWINGS

Drawing Figures

FIG. 1 shows a typical single tube stethoscope.

FIG. 2 shows a typical double tube stethoscope.

FIG. 3 shows a single tube stethoscope with device integrated into acoustic tubing.

FIG. 4 shows a double tube stethoscope with device integrated into acoustic tubing.

FIG. 5 shows a single tube stethoscope retrofitted with device.

FIG. 6 shows a double tube stethoscope retrofitted with device.

FIG. A-A shows a cross section of the device integrated into a single tube stethoscope.

FIG. B-B shows a cross section of the device integrated into a double tube stethoscope.

FIG. C-C shows a cross-section of a retrofit to an existing single tube stethoscope.

FIG. D-D shows a cross-section of a retrofit to an existing double tube stethoscope.

### DETAILED DESCRIPTION

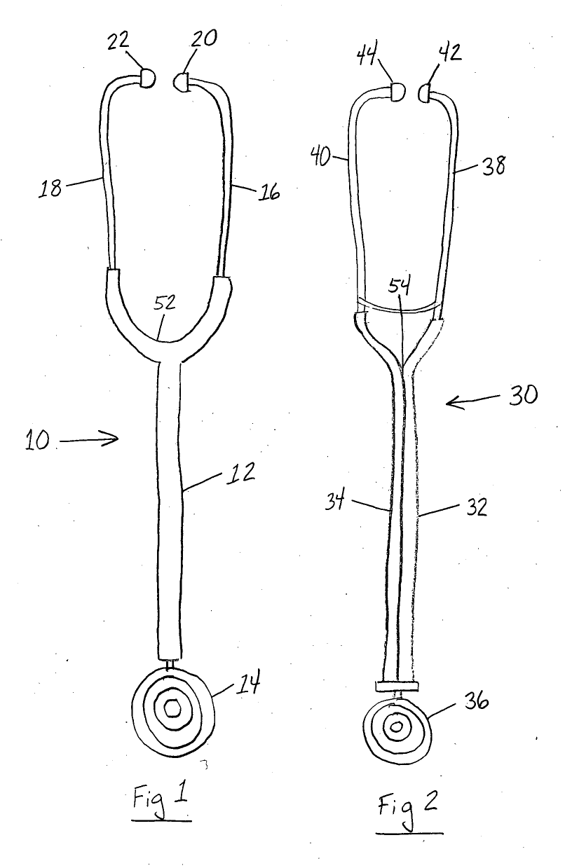

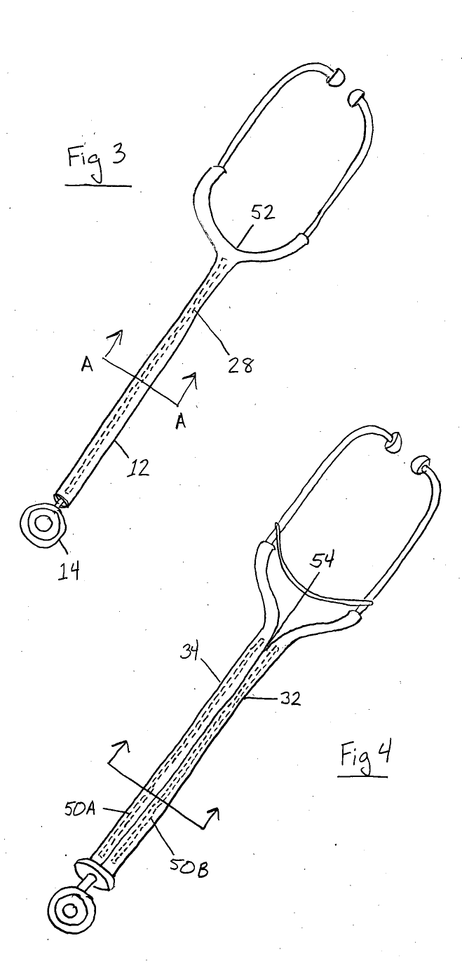

Description—FIGS. 1 and 2

Typical stethoscopes are shown in FIGS. 1 and 2.

FIG. 1 depicts a typical single tube stethoscope 10 with a single sound tube 12 connecting a chestpiece 14 to binaural eartubes 16 and 18, which terminate in eartips 20 and 22. Sound tube 12 branches at Y-shaped juncture 52.

FIG. 2 depicts a typical double tube stethoscope 30 with sound tubes 32 and 34 connecting a chestpiece 36 to binaural eartubes 38 and 40, which terminate in eartips 42 and 44. Sound tubes 32 and 34 branch at Y-shaped juncture 54.

FIGS. 3 and A-A—Preferred Embodiment

A preferred embodiment is shown in FIGS. 3 (isometric) and A-A (cross section). A metal wire 28 is embedded in sound tube 12 and runs approximately the entire length of sound tube 12, from Y-shaped juncture 52 to just before sound tube 12 terminates at chestpiece 14. It is preferable that wire 28 not protrude internally or externally at any time from sound tube 12. Wire 28 may run parallel to the length of sound tube 12 or may follow a helical path about acoustic conduit 56. Wire 28 has a semi-rigid quality such that it can be bent to conform to a desired shape and will hold said shape until bent to a new shape.

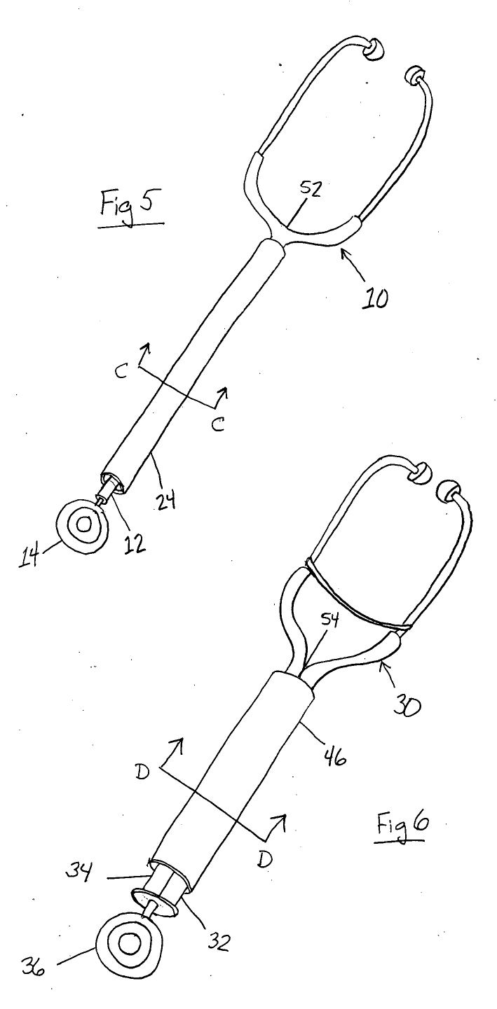

FIGS. 4 and B-B—Additional Embodiment

An additional embodiment is shown in FIGS. 4 (isometric) and B-B (cross section). Metal wires 50A and 50B are embedded in sound tubes 32 and 34 and run approximately the entire length of sound tubes 32 and 34, from Y-shaped juncture 54 to just before sound tubes 32 and 34 terminate at chestpiece 36. It is preferable that wires 50A and SOB not protrude internally or externally at any time from sound tubes 32 and 34. Wires 50A and 50B may run parallel to the length of sound tubes 32 and 34 or may follow individual helical paths about acoustic conduits 58 and 60. Wires 50A and 50B have a semi-rigid quality such that they can be bent to conform to a desired shape and will hold said shape until bent to a new shape.

FIG. 5 and C-C—Additional Embodiment

An additional embodiment is illustrated in FIGS. 5 (isometric view) and C-C (cross section). Sleeve 24 encompasses sound tube 12 of typical single tube stethoscope 10. A metal wire 26 is embedded in and runs approximately the entire length of sleeve 24, from just before each endpoint of sleeve 24. It is preferable that wire 26 not protrude internally or externally at any time from sleeve 24. The overall length of sleeve 24 is ideally slightly less than the length of sound tube 12, from Y-shaped juncture 52 to just before chestpiece 14. Wire 26 may run parallel to the length of sleeve 24 or may follow a helical path about interior conduit 62. Wire 26 has a semi-rigid quality such that it can be bent to conform to a desired shape and will hold said shape until bent to a new shape. During installation, chestpiece 14 is temporarily removed to allow sleeve 24 to be slid over and around sound tube 12. Interior conduit 62 is large enough to allow sleeve 24 to slide over sound tube 12 during installation but not so large as to allow sleeve 24 to “float” loosely about sound tube 12. Sleeve 24 is preferably made of material typical in the flexible tubing of common hospital stethoscopes, must be impermeable to bodily fluids, and must lend itself to be easily and effectively sterilized per standard medical procedure.

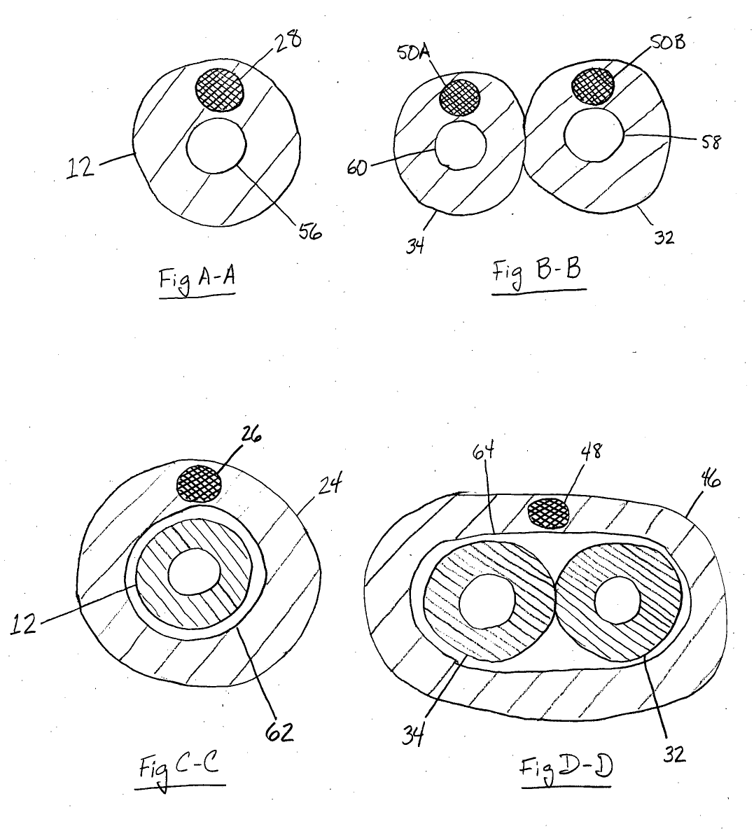

FIG. 6 and D-D—Additional Embodiment

An additional embodiment is illustrated in FIGS. 6 (isometric view) and D-D (cross section). Sleeve 46 encompasses sound tubes 32 and 34 of typical double tube stethoscope 30. A metal wire 48 is embedded in and runs approximately the entire length of sleeve 46, from just before each endpoint of sleeve 46. It is preferable that wire 48 not protrude internally or externally at any time from sleeve 46. The overall length of sleeve 46 is ideally slightly less than the length of sound tubes 32 and 34, from Y-shaped juncture 54 to just before chestpiece 36. Wire 48 may run parallel to the length of sleeve 46 or may follow a helical path about interior conduit 64. Wire 48 has a semi-rigid quality such that it can be bent to conform to a desired shape and will hold said shape until bent to a new shape. During installation, chestpiece 36 is temporarily removed to allow sleeve 46 to be slid over and around sound tubes 32 and 34. Interior conduit 64 is large enough to allow sleeve 46 to slide over sound tube 32 and 34 during installation but not so large as to allow sleeve 46 to “float” loosely about sound tubes 32 and 34. Sleeve 46 is preferably made of material typical in the flexible tubing of common hospital stethoscopes, must be impermeable to bodily fluids, and must lend itself to be easily and effectively sterilized per standard medical procedure.

Advantages

From the description above, a number of advantages of my shape-retaining stethoscope tubing become evident:

(a) The wire (or wires) embedded in the stethoscope tubing or sleeve allows the stethoscope tubing to retain a desired shape during use.

(b) The invention may be incorporated into the manufacture of new stethoscopes of both the single tube and double tube variety or may be retrofit on stethoscopes of both types.

(c) The shape-retaining quality of the invention allows users to bend the stethoscope around their necks where it will hold its position without falling or swinging.

Operation—FIGS. 3, 4, 5, 6

The manner of using the shape-retaining stethoscope tubing is to bend tubing 12 (FIG. 3) or 32 and 34 (FIG. 4) to the desired shape for placement of stethoscope chestpiece 14 or 36 under the clothing of the person to be examined. This is ideally accomplished by:

(1) locating a suitable opening (such as a sleeve, neck, or button flap) in the clothing of the patient;

(2) determining the desired position of the chestpiece on the patient's body (such as the chest, abdomen, or back);

(3) bending the stethoscope tubing to a shape which will allow the chestpiece to enter the opening in the clothing, be routed under the clothing, and ultimately reach the ideal position on the patient's body; and

(4) placing the chestpiece into the opening in the clothing and pushing on the stethoscope tubing until the chestpiece arrives at the desired location.

If necessary, the stethoscope user's hand can apply pressure on the chestpiece from outside of the clothing to assure firm contact with patient's body.

To use the retrofit version of the invention, chestpiece 14 of typical single tube stethoscope 10 (FIG. 5) or chestpiece 36 of typical double tube stethoscope 30 (FIG. 6) is removed, sleeve 24 or 46 is slid over existing single sound tube 12 or double sound tubes 32 and 34, and the chestpiece replaced. Once the sleeve is in place, operation is identical to steps one through four above.

To store the stethoscope, when not in use, around the user's neck, the acoustic tubing is bent to a shape which will allow the stethoscope to grip the neck, preventing the stethoscope from falling or swinging uncontrollably as the user walks, bends over, etc.

Conclusions, Ramifications, and Scope

Accordingly, the reader will see that the shape-retaining stethoscope tubing of this invention can be used to quickly, flexibly and accurately examine a patient without exposing the patient to unnecessary clothing removal and the potential risks involved. In addition, this invention can be incorporated into the manufacture of new stethoscopes or can be retrofit onto existing stethoscopes. Furthermore, the shape-retaining quality of the invention allows the stethoscope to be stored about the neck of the user and will not fall or swing undesirably.

Although the description above contains many specificities, these should not be construed as limiting the scope or spirit of the invention but as merely providing illustrations of some of the presently preferred embodiments of this invention. For example, more wires may be embedded into the tubing or sleeve; a material other than metal may be used for the wire(s); the shape and material of the retrofit sleeve may be changed; the invention may be used for non-medical purposes, etc.

Thus the scope of the invention should be determined by the appended claims and their legal equivalents, rather than by the examples given.

I claim:

1. A stethoscope tubing comprising:
a. flexible, acoustic tubing, and
b. means for allowing said tubing to temporarily retain a desired shape.

2. The stethoscope tubing of claim 1 wherein said means is wire embedded in said acoustic tubing.

3. The stethoscope tubing of claim 2 wherein said wire is approximately the same length as said acoustic tubing.

4. The stethoscope tubing of claim 3 wherein said wire is embedded such that it traverses a path parallel to interior conduit of said acoustic tubing.

5. The stethoscope tubing of claim 3 wherein said wire is embedded such that it follows a helical path about interior conduit of said acoustic tubing.

6. A sleeve for retrofit of stethoscopes comprising:
a. flexible tubing, and
b. means for allowing said tubing to temporarily retain a desired shape.

7. The sleeve of claim 6 wherein said flexible tubing has an interior opening of sufficient size so as to allow said tubing to encompass the acoustic tubing of a typical hospital stethoscope.

8. The sleeve of claim 7 wherein said wire is approximately the same length as said flexible tubing.

9. The sleeve of claim 8 wherein said wire is embedded such that it traverses a path parallel to interior conduit of said flexible tubing.

10. The sleeve of claim 8 wherein said wire is embedded such that it follows a helical path about interior conduit of said flexible tubing.
